SEQUENCE LISTING (1 sequence)

SEQ ID NO: 1 - DNA, 1516 bp, Homo Sapiens
acaagctccctgctgatggacatttgtctttgttactatgatagtaatataatcaacatttatatatgttttgtatgtatctataatacacatgcacatacacatgcatatttctgcagggatagccatagtaaataactagtaacggtattgcaagttaaaggaacaatctcattgcttgaaattttaaattttgaaatacactgccaattttcatggtctctccttgtaagctagtttgggctttctcacagcatgacaggctcagggcagtcagaccatcctggccaaagagcagagtgccacagaccacaactgcttctaatcagccatcttcccaaagccttctcttttttctattaataactttgtatgagattccatcttaatacttttctgttgtttggtcttgtaagagcttatttttcctgaaccaggaagtggttcagggcggtttttctaacttcacagagctccctcttctgttagcttttgtgaaatggtcaaaaacatagcagcctgccttctgagttctccatcccaccctggttgggccttctctatccttgtctgtgttgtttatatcctgctgaagtgtgattccacttgtggcagtttctcctctgtgtaggatcaaaagggctgtgactggttggtttgaaaatttcttataccctagactattccagtgcctttcagaagtttccaaggccctctcacactaatctattatcatattgggcaaaactccttgcagtttcagctactattccctgattgacttttcagtaaatctatctctcagtctttcagtatccaaagaagattggttctaggaccaccatcccgctgcctccacagataccaaaatcagaggatgctcaattccctcttataaaacgttgcagtatttgcatataatctgcacatgtatttctgtatattttaaatcatccctagattacttataatacctgatacaatataaatgctaaatagctgtaacactgtatctttaaaatttacattattttttgttgttgtattattatttttattgtatttttaaaaaatattttccatctacagtcagtagaatccacggatacagaacctatggataggaaggaccaactgtatcttttagtgttttgaggttcttgaattctcaggtcgtttgctttcctttgctttctcccaagtcttgttttacaatttgctttagtcattcactgaaactttaaaaaacattagaaaacctcacagtttgtaaatctttttccctattatatatatcataagataggagcttaaataaagagttttagaaactactaaaatgtaaatgacataggaaaactgaaagggagaagtgaaagtgggaaattcctctgaatagagagaggaccatctcatataaataggccatacccatggagaaaggacattctaactgcaacctttcgaagcctttgctctggcacaacaggtagtaggcgacactgttcgtgttgtc

(19) United States
(12) Patent Application Publication
(10) Pub. No.: US 2021/0364497 A1
(43) Pub. Date: Nov. 25, 2021

(54) IFN-beta Reporter System for Primary Cells

(71) Applicant: UNIVERSITY OF MIAMI, MIAMI, FL (US)

(72) Inventor: GLEN N BARBER, MIAMI, FL (US)

(73) Assignee: UNIVERSITY OF MIAMI, MIAMI, FL (US)

(21) Appl. No.: 17/044,673

(22) PCT Filed: Apr. 2, 2019
(86) PCT No.: PCT/US2019/025380

§ 371 Date: Oct. 1, 2020

Related U.S. Application Data
(60) Provisional application No. 62/651,486, filed on Apr. 2, 2018.

(51) Int. Cl. G01N 33/50

(52) U.S. Cl. CPC G01N 33/5023; G01N 2333/916; G01N 2333/90241; G01N 2333/565

(57) ABSTRACT

The present disclosure relates, in general, to methods for screening for modulators of IFNβ activity using a GLuc/SEAP dual reporter system.

This application is a (i) U.S. National Phase Application of PCT/US2019/025380, filed Apr. 2, 2019, which claims the priority benefit of (ii) U.S. Provisional Application No. 62/651,486, filed Apr. 2, 2018, 62/648,096, the disclosure of (i) and (ii) are incorporated herein by reference in their entireties.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file STNG-01003US1_ST25.TXT, created Sep. 10, 2020, 2,426 bytes, machine format IBM-PC, MS-Windows operating system, is hereby incorporated by reference.

### FIELD OF THE DISCLOSURE

The present disclosure relates, in general, to methods for screening for modulators of IFNβ activity using a GLuc/SEAP dual reporter system.

### BACKGROUND

Reporter gene assays were developed to analyze the genetic regulatory elements that control the expression of genes of interest or determine what factors play a role in modulating gene expression. In the reporter assay, the regulatory elements of the gen of interest control the expression of the reporter gene itself, and therefore reporter expression can be correlated with the activity of the regulatory elements. Reporter genes commonly used in gene expression assays include β-galactosidase (lacZ), chloramphenyl acetyltransferase (CAT), β-glucuronidase (GUS) and fluorescent proteins (green, yellow or red fluorescent protein [GFP, YFP or RFP, respectively), luciferase, and secretory alkaline phosphatase (SEAP).

Luciferase is a naturally luminescent protein expressed by various firefly species, including. A summary of different luciferase enzymes can be found via ThermoFisher Scientific (Waltham, Mass.) and are well known in the field of reporter gene assays. Luciferases enzymes from species such as Gaussia, Metridia and Cypridina are naturally secreted and are frequently used to study proteins and genes in secretory pathways.

Current luciferase promoter reporter systems are mostly established in transformed cell lines with abnormal cellular functions which can lead to inaccurate determination of protein or gene function.

Methods of using IFNβ as a reporter gene are described in US20130039933 (Barber et al.) which discloses a Luciferase (Luc) reporter construct comprising the IFNβ promoter and is transfected in 293T cells.

### SUMMARY

The present reporter system is established in primary immortalized hTERT cell with intact cellular function and innate immunity, which will facilitate accurate evaluation of IFNβ promoter in normal cellular environment. This system is improved over available reporter systems because it is effective in primary cell lines that have not had their function altered by transformation.

The GLuc/SEAP-promoter reporter system is established in primary human cells that have intact cellular signaling pathways and innate immune response, which is essential for screening activators and/or inhibitors that affect cellular function in innate immunity. The secretable Alkaline phosphatase (SEAP) provides an additional internal control which will facilitate normalization and statistical evaluation of promoter activity.

In various embodiments, the disclosure provides a method of screening for an agent that modulates IFNβ activity comprising contacting a cell comprising a reporter construct, the construct comprising an IFNβ promoter sequence and a Gaussia Luciferase (GLuc) and Secreted Alkaline Phosphatase (SEAP) dual reporter system, with a candidate IFNβ modulating agent, the method comprising: measuring the GLuc/SEAP expression before and after contact of the cell with the candidate modulator, and identifying the candidate modulator as an activator or inhibitor of IFNβ based on a change in ratio of expression of GLuc/SEAP from the reporter construct.

In various embodiments, the construct is a plasmid. In various embodiments, the construct is a lentiviral construct, retroviral construct or plasmid-based construct. In some embodiments, the plasmid is pEZX-LvPG04.

In various embodiments, the cell is selected from the group consisting of hTERT cells, immortalized stem cells, Vero cells, HEK293 cells, and normal human cells. Additional cells contemplated are described in more detail in the Detailed Description.

In various embodiments, the candidate agent is single-stranded RNA, single-stranded DNA, double-stranded DNA, double-stranded RNA, microRNA, a protein, a peptide, a virus, a bacteria, a fungus, a parasite or a small molecule.

In various embodiments on the method, an increase in the ratio of GLuc/SEAP indicates the candidate modulator is an activator of IFNβ. In various embodiments, a decrease in the ratio of GLuc/SEAP indicates the candidate modulator is an inhibitor of IFNβ.

In various embodiments, the construct further comprises a gene for a selectable marker. In various embodiments, the selectable marker is selected from the group consisting of puromycin, neo, 5FOA, hygromycin, G418, and bleomycin.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE FIGURES

FIG. 1 sets out the nucleotide sequence for the IFNβ promoter.

FIG. 2 illustrates the transfection of hTERT primary cells with the IFNβ reporter construct.

FIGS. 3A-3B show the effects of dsDNA, 2′-3′ cGAMP and LPS1 (FIG. 3A) or dsDNA, 2′-3′ cGAMP and poly I:C (FIG. 3B) on hTERT cells transfected with the IFNβ reporter system.

### DETAILED DESCRIPTION

Current luciferase promoter reporter systems are mostly established in transformed cell lines with abnormal cellular functions. The present reporter system is established in primary immortalized hTERT cell with intact cellular function and innate immunity, which will facilitate accurate evaluation of IFNβ promoter in normal cellular environment.

### Definitions

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P-NH2) or a mixed phosphoramidate-phosphodiester oligomer. Peyrottes et al. (1996) Nucleic Aids Res. 24: 1841-8; Chaturvedi et al. (1996) Nucleic Acids Res. 24:2318-23; Schultz et a. (1996) Nucleic Acids Res. 24: 2966-73. A phosphorothioate linkage can be used in place of a phosphodiester linkage. Braun et al. (1988) J. Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NO) also includes the complement sequence.

The following are non-limiting examples of polynucleotides: a gene or gene fragment, exons, introns, genomic RNA, mRNA, tRNA, rRNA, ribozymes, cDNA, microRNA, recombinant polynucleotides, branched polynucleotides, plasmids, vectors, isolated DNA of any sequence, isolated RNA of any sequence, nucleic acid probes, and primers. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

“Under transcriptional control” is a term well understood in the art and indicates that transcription of a polynucleotide sequence depends on its being operably (operatively) linked to an element which contributes to the initiation of, or promotes, transcription. “Operably linked” refers to a juxtaposition wherein the elements are in an arrangement allowing them to function.

A “host cell” includes an individual cell or cell culture which can be or has been a recipient of a vector(s) described herein. Host cells include progeny of a single host cell, and the progeny may not necessarily be completely identical (in morphology or in total DNA complement) to the original parent cell due to natural, accidental, or deliberate mutation and/or change. A host cell includes cells transfected, transduced, transformed or infected in vivo or in vitro with a vector herein.

A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Peptides” or “oligopeptides” are short amino acid sequences, typically between 3 and 100 amino acid residues in length and encompass naturally occurring amino acid residues and non-naturally occurring analogs of residues which may be used singly or in combination with naturally occurring amino acid residues in order to give the peptide a particular conformational specificity or a particular biological activity, such as resistance to proteolysis. Peptides include repeats of peptide sequences and may include 2, 3, 4, 5, 6, 7, 8, 9, 10 or more copies of an amino acid sequence arranged head-to-tail or head-to-head. Peptides may be conjugated to non-peptidic moieties. Peptides include dimers, trimers or higher order multimers, e.g. formed through conjugation to other polymeric or non-polymeric moieties, such as PEG.

“Polypeptides” are longer amino acid sequences, typically 100 or more amino acid residues in length, and encompass naturally occurring amino acid residues and non-naturally occurring analogs of residues which may be used singly or in combination with naturally occurring amino acid residues.

A “small” molecule or “small” organic molecule as used herein refers to a non-polymeric organic chemical compound having a molecular weight of about 1000 Daltons or less.

### IFNβ

Human INF-beta sequences are described in U.S. Pat. No. 5,908,626 and Fiers et al. (1982) Philos. Trans. R. Soc. Lond., B. Biol. Sci. 299:29-38) and has been deposited with GenBank under Accession No. M25460. IFNs are comprised of two groups referred to as type I (α/β) and type II (γ). IFN-α/β genes include several a genes and a single β gene, are clustered onto the short arm of human chromosome 9, and are expressed from most cell types (Obuchi et al., J Virol. 77(16): 8843-8856, 2003). IFN-γ is encoded by a single gene on chromosome 12 and is mainly secreted by Th-1 lymphocytes and natural killer (NK) cells.

The expression of the IFN-α/βs can be induced by a number of stimuli, including viral infection, double-stranded RNA and lipopolysaccharides. It is thought that RNA species arising from invading viruses trigger signaling cascades, involving the NF-κB and IFN regulatory factor 3 (IRF-3) pathways, that lead to the transcriptional activation of I FN-β, which is then secreted. IFN-α/βs bind to species-specific cell surface receptors and trigger the activation of the Janus protein kinases (specifically JAK-1 and TYK2) and the signal transducer and activator of transcription (STAT 1 and STAT2) pathway (Ihle, et al. Curr. Opin. Cell Biol. 13:211-217, 2001). In conjunction with p48 (ISGF3/IRF-9), phosphorylated STAT1 and STAT2 heterodimers bind to cognate DNA recognition motifs referred to as IFN-stimulated response elements, which are located in the promoter regions of numerous genes. This event invokes transcriptional activation of target genes such as the double-stranded RNA-dependent protein kinase PKR, the death ligand TRAIL, 2′-5′ oligoadenylate/RNAse L proteins, heat shock proteins, major histocompatibility class antigens, PML, STAT 1, and IRF-7, among many others (Der et al., Proc. Natl. Acad. Sci. USA 95:15623-15628, 1998; Stark, et al. Annu. Rev. Biochem. 67:227-264, 1998). In addition to being able to potently prevent virus replication through intracellular mechanisms, the IFNs are known as important modulators of the immune system and are able to activate NK and T cells and facilitate the maturation of professional antigen-presenting cells such as dendritic cells (DC) (Kadowaki, et al., J. Exp. Med. 192:219-226, 2000).

Candidate modulators of IFNβ include activators of the Toll-Like Pathway (TLR), Ri\IG-I/MDA5 pathway or cGAS/STING pathway or other pathways that activate the type I interferon pathway such as nucleic acids, such as single- or double-stranded DNA and/or RNA, and microRNA; proteins or peptides; microbial agents, such as virus, lipopolysaccharides, bacteria, parasite or fungus; or, small molecule agents cyclic dinucleotides.

### Host Cells

The present invention also provides host cells comprising (i.e., transformed, transfected or infected with) the vectors or constructs described herein. Exemplary cells include mammalian cells. Primary cells, or in other examples, immortalized or tumor cell lines can be used. A number of cell lines commonly known in the art are available for use. By way of example, such primary cell lines include, but are not limited to, hTERT, HEK293, monkey Vero cells, immortalized stem cells. A database of immortalized cells is compiled by Applied Biological Materials Inc., (Richmond BC, Canada) and include, immortalized, adipose cells, blood, brain, breast, bone marrow, colon, lung, heart, liver, skin, spleen, stem cells, and others. It is contemplated that these cells lines are useful in the present methods.

Additional cell lines known in the art include BHK (baby hamster kidney) cells, CHO (Chinese hamster ovary) cells, HeLA (human) cells, mouse L cells, ESK-4, PK-15, EMSK cells, MDCK (Madin-Darby canine kidney) cells, MDBK (Madin-Darby bovine kidney) cells, Jurkat T cells, and Hep-2 cells. Such cell lines are publicly available for example, from the ATCC and other culture depositories.

### Reporter System

GeneCopoeia's SECRETE-PAIR™ Dual Luminescence Assay Kit is designed to analyze the activities of Gaussia Luciferase (GLuc) and Secreted Alkaline Phosphatase (SEAP) in a dual-reporter system. Both GLuc and SEAP are secreted reporter proteins, permitting detection without cell lysis. Secrete-Pair measures dual reporter signals and allows transfection normalization (See GeneCopoeia product description). The GLuc reporter provides a reporter system to detect up or down regulation of the reporter gene while the secondary reporter, secreted Alkaline Phosphatase (SEAP), serves as an internal control. The dual-reporter system enables transfection normalization for cross-sample comparison.

In various embodiments, the GLuc/SEAP reporter is expressed in a construct, e.g., a plasmid or other expression vector, for transfection or transduction into a host cell for expression. In various embodiments, the reporter is expressed in a viral vector, such as a lentiviral, adenoviral, AAV, retroviral, baculoviral, or other viral vector commonly used in the art for gene expression studies. In one embodiment, the construct is a plasmid, wherein the plasmid is the pEZX-LvPG04 lentiviral vector. Other expression vectors including plasmids, plasmid-based vectors or retroviruses that express secreted on non-secreted luciferase or other reporter such as GFP are also contemplated.

It is further contemplated that the construct may comprise a gene for a selectable marker. Selectable markers are well-known in the field of molecular biology and gene expression and are useful for selection of cells that express the construct or vector that is transfected into the cells during culture. Selection methods allow for preferential growth of cells that express the construct of interest. Many selectable markers for eukaryotic culture are directed to antibiotic resistance genes, or toxicity resistance genes. Exemplary selectable markers include puromycin, neo, 5FOA, hygromycin, G418, and bleomycin. Genes that confer resistance to these selection markers are known in the art and readily obtainable by those practicing in the field.

### Kits

The present disclosure also provides kits comprising the reporter construct described herein. In one embodiment, such a kit includes a compound or composition described herein (e.g., a composition comprising a reporter construct as described herein, and optionally host cells), packaged in a container such as a sealed bottle or vessel, with a label affixed to the container or included in the package that describes use of the compound or composition in practicing the method. In one embodiment, the composition is packaged in a unit dosage form. Preferably, the kit contains a label that describes use of the compositions.

Additional aspects and details of the disclosure will be apparent from the following examples, which are intended to be illustrative rather than limiting.

### Examples

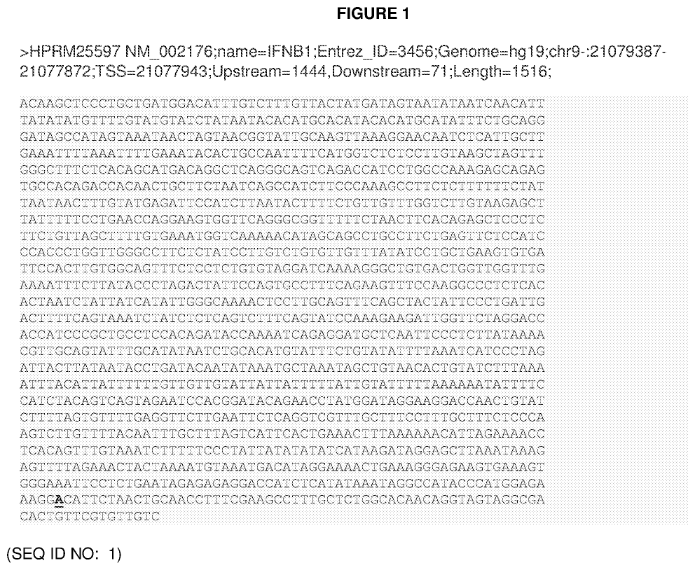

Human IFNβ promoter (sequence in FIG. 1) was cloned into Lentiviral vector pEZX-LvPG04 at GeneCopoeia. Lentiviral plasmid was co-transfected with LentiViral Package System (LENT-PACT™) into 293T cells using Lipofectamine 2000. 293T medium containing Lentiviral particles was collected 48 hours post transfection, filtered through 0.2 μM filter, and transduced to pre-seeded hTERT cells in the presence of 10 μg/ml polybrene. Transduction was repeated twice in 2 consecutive days. 48 hours after the last transduction, hTERT cells were selected by 2 μg/ml puromycin for 4 weeks. Single colonies of transduced hTERT cells were puromycin selected, and expanded for IENβ promoter-luciferase assay validation.

To test the establishment of GLuc/SEAP-ρIENs1 reporter system, hTERT clones were mock treated (lipo) or transfected with synthetic double-stranded DNA (dsDNA). hTERT-ρIENβ-GLuc cells were transfected with synthetic 90-mer double-stranded DNA (dsDNA90) at 3 μg/ml using Lipofectamine 2000. 24 hours later, cell media was collected and analyzed for GLuc and SEAP activity using the Secrete-Pair Dual Luminescence Assay Kits (GeneCopoeia).

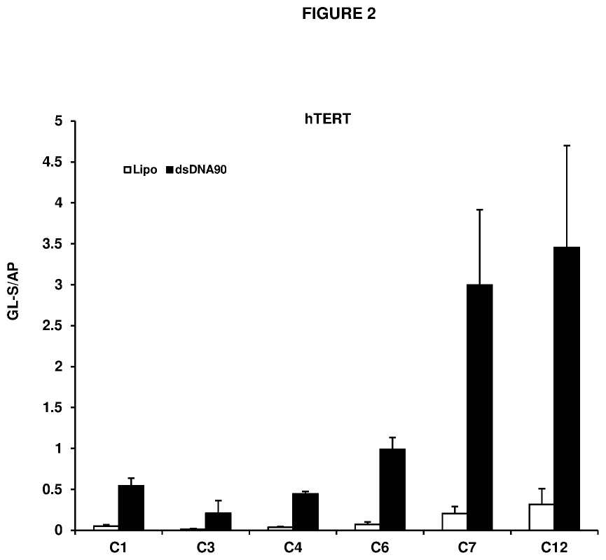

Results are shown in Tables 1-3, and FIG. 2, and refer to transfectant clone number and Round 1 or Round 2 of transfection measured in Relative Light Units (RLU).

TABLE 1
GL-s (RLU/s)

| | |
|---|---|
| R1 | R2 |

| | Lipo | dsDNA90 | Lipo | dsDNA90 |
|---|---|---|---|---|
| C1 | 384 | 3987 | 626 | 3776 |
| C3 | 152 | 2070 | 386 | 4224 |
| C4 | 624 | 6158 | 859 | 5971 |
| C6 | 1024 | 9378 | 1428 | 8144 |
| C7 | 995 | 8639 | 1737 | 13512 |
| C12 | 1196 | 9382 | 2748 | 11949 |

TABLE 2
AP

| | |
|---|---|
| R1 | R2 |

| | Lipo | dsDNA90 | Lipo | dsDNA90 |
|---|---|---|---|---|
| Cl | 10191 | 8333 | 10209 | 6199 |
| C3 | 24603 | 21351 | 20055 | 13284 |
| C4 | 22619 | 14511 | 19446 | 12789 |
| C6 | 21476 | 10564 | 15388 | 7463 |
| C7 | 6941 | 3679 | 6585 | 3705 |
| C12 | 6543 | 3643 | 6082 | 2756 |

TABLE 3
GL-s/AP Ratio

| | |
|---|---|
| R1 | R2 |

| | Lipo | dsDNA90 | Lipo | dsDNA90 |
|---|---|---|---|---|
| C1 | 0.03768 | 0.478459 | 0.061318 | 0.609131 |
| C3 | 0.006178 | 0.096951 | 0.019247 | 0.317977 |
| C4 | 0.027587 | 0.424368 | 0.044174 | 0.466886 |
| C6 | 0.047681 | 0.887732 | 0.0928 | 1.09125 |
| C7 | 0.143351 | 2.348192 | 0.263781 | 3.646964 |
| C12 | 0.182791 | 2.57535 | 0.451825 | 4.335631 |

A high increase in normalized GLuc/SEAP ratio represents activation of the IPNβ1 promoter upon dsDNA stimulation. The experiments show that this reporter system is a viable means by which to measure IFNβ stimulation in response to dsDNA stimulation or other stimulation of the IFNβ promoter in primary cells.

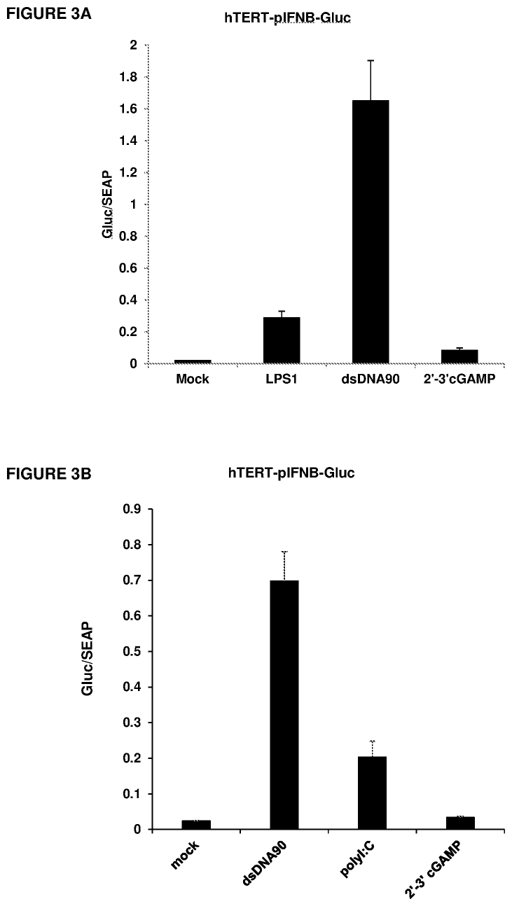

In additional experiments, hTERT-pIFNB-Gluc cells were treated with LPS1 (1 μg/ml) or transfected with dsDNA90 (3 μg/ml) or 2′-3′ cGAMP (3 μg/ml) for 24 hours. Culture media were collected and analyzed for Gaussia Luciferase (Gluc) activity and normalized by Secreted Alkaline Phosphatase (SEAP) internal control. FIG. 3A shows that GLuc expression is activated by both dsDNA and LPS1, and to a small extent by 2′-3′ cGAMP.

hTERT-pIFNB-Gluc cells were then treated with poly:C (3 μg/ml), dsDNA90 (3 μg/ml), or 2′-3′ cGAMP (3 μg/ml) for 24 hours. Culture media were collected and analyzed for Gaussia Luciferase (Gluc) activity and normalized by Secreted Alkaline Phosphatase (SEAP) internal control. FIG. 3B shows that GLuc expression is activated by both dsDNA and poly I:C and to a small extent by 2′-3′ cGAMP.

These results suggest that the assay herein is activated by dsRNA/poly IC (RIG-I/MDA5 and TLR pathway), lipopolysaccharide (TLR4) and dsDNA (STING) pathway. Thus, the present modified cell line could be used to look for activators of IFN beta that function through a number of key innate immune pathways. For example the cell line could be useful in identifying TLR activators, or RIG-1 activators or STING activators.

Numerous modifications and variations of the invention as set forth in the above illustrative examples are expected to occur to those skilled in the art. Consequently only such limitations as appear in the appended claims should be placed on the invention.

What is claimed:

1. A method of screening for an agent that modulates IFN-β activity comprising:
(a) contacting a primary cell line with a dual reporter construct comprising:
an IFN-β promoter sequence;
a Gaussia Luciferase (GLuc) reporter construct; and
a Secreted Alkaline Phosphatase (SEAP) reporter construct;
(b) measuring a GLuc expression of the dual reporter construct;
(c) measuring a SEAP expression of the dual reporter construct;
(d) calculating a first ratio of expression of the GLuc expression to the SEAP expression from the measurements in step (b) and step (c);
(e) contacting the dual reporter construct with the agent;
(f) measuring the GLuc expression of the dual reporter construct after contacting the primary cell line with the agent;
(g) measuring the SEAP expression of the dual reporter construct after contacting the primary cell line with the agent;
(h) calculating a second ratio of expression of the GLuc expression to the SEAP expression from the measurements in step (f) and step (g); and
(i) identifying the agent as an activator of IFN-β based on an increase in the second ratio of expression calculated in step (h) compared to the first ratio of expression calculated in step (d).

2. The method of claim 1, further comprising identifying the agent as an inhibitor of IFN-β based on a decrease in the second ratio of expression calculated in step (h) compared to the first ratio of expression calculated in step (d).

3. The method of claim 1, where the agent is selected from the group consisting of a single-stranded RNA, a single-stranded DNA, a double-stranded DNA, a double-stranded RNA, a microRNA, a protein, a peptide, a virus, a bacteria, a fungus, a parasite or a small molecule.

4. The method of claim 1, where the primary cell line is normal human cells.

5. The method of claim 1, where the dual reporter construct is a plasmid.

6. The method of claim 5, where the plasmid is pEZX-LvPG04.

7. The method of claim 1, where the dual reporter construct is selected from the group consisting of a lentiviral construct, a retroviral construct and a plasmid-based construct.

8. The method of claim 7, where the dual reporter construct is plasmid pEZX-LvPG04.

9. The method claim 1, where the dual reporter construct further comprises a gene for a selectable marker selected from the group consisting of puromycin, neo, 5FOA, hygromycin, G418, and bleomycin.

10. A method of screening for an agent that modulates IFN-β activity comprising:
(a) contacting a cell with a dual reporter construct comprising:
an IFN-β promoter sequence;
a gene for a selectable marker selected from the group consisting of puromycin, neo, 5FOA, hygromycin, G418, and bleomycin;
a Gaussia Luciferase (GLuc) reporter system; and
a Secreted Alkaline Phosphatase (SEAP) reporter system;
(b) detecting cells that have taken up the selectable marker and are expressing the dual reporter construct;
(c) measuring a GLuc expression of the dual reporter construct;
(d) measuring a SEAP expression of the dual reporter construct;
(e) calculating a first ratio of expression of the GLuc expression to the SEAP expression from the measurements in step (c) and step (d);
(f) contacting the dual reporter construct with the agent;
(g) measuring the GLuc expression of the dual reporter construct after contacting the cell with the agent;
(h) measuring the SEAP expression of the dual reporter construct after contacting the cell with the agent;
(i) calculating a second ratio of expression of the GLuc expression to the SEAP expression from the measurements in step (g) and step (h); and
(j) identifying the agent as an activator of IFN-β based on an increase in the second ratio of expression calculated in step (i) compared to the first ratio of expression calculated in step (e).

11. The method of claim 10, further comprising identifying the agent as an inhibitor of IFN-β based on a decrease in the second ratio of expression calculated in step (i) compared to the first ratio of expression calculated in step (e).

12. The method of claim 10, where the agent is selected from the group consisting of a single-stranded RNA, a single-stranded DNA, a double-stranded DNA, a double-stranded RNA, a microRNA, a protein, a peptide, a virus, a bacteria, a fungus, a parasite or a small molecule.

13. The method of claim 10, where the cell is selected from the group consisting of hTERT cells, immortalized stem cells, Vero cells, HEK293 cells, and normal human cells.

14. The method of claim 10, where the dual reporter construct is plasmid pEZX-LvPG04.

15. The method of claim 10, where the dual reporter construct is selected from the group consisting of a lentiviral construct, a retroviral construct and a plasmid-based construct.

16. The method of claim 15, where the plasmid-based construct is pEZX-LvPG04.

17. A method of screening for an agent that modulates IFN-β activity comprising:
(a) contacting a primary cell line with a dual reporter construct comprising:
an IFN-β promoter sequence;
a gene for a selectable marker selected from the group consisting of puromycin, neo, 5FOA, hygromycin, G418, and bleomycin;
a Gaussia Luciferase (GLuc) reporter system; and
a Secreted Alkaline Phosphatase (SEAP) reporter system;
(b) detecting cells that have taken up the selectable marker and are expressing the dual reporter construct;
(c) measuring a GLuc expression of the dual reporter construct;
(d) measuring a SEAP expression of the dual reporter construct;
(e) calculating a first ratio of expression of the GLuc expression to the SEAP expression from the measurements in step (c) and step (d);
(f) contacting the dual reporter construct with the agent;
(g) measuring the GLuc expression of the dual reporter construct after contacting the primary cell line with the agent;
(h) measuring the SEAP expression of the dual reporter construct after contacting the primary cell line with the agent;
(i) calculating a second ratio of expression of the GLuc expression to the SEAP expression from the measurements in step (g) and step (h); and
(j) identifying the agent as an activator of IFN-β based on an increase in the second ratio of expression calculated in step (i) compared to the first ratio of expression calculated in step (e); or
(k) identifying the agent as an inhibitor of IFN-β based on a decrease in the second ratio of expression calculated in step (i) compared to the first ratio of expression calculated in step (e).

18. The method of claim 17, where the agent is selected from the group consisting of a single-stranded RNA, a single-stranded DNA, a double-stranded DNA, a double-stranded RNA, a microRNA, a protein, a peptide, a virus, a bacteria, a fungus, a parasite or a small molecule.

19. The method of claim 17, where the primary cell line is a normal human cell line.

20. The method of claim 17, where the dual reporter construct is a plasmid, where the plasmid is pEZX-LvPG04.
